(19) United States
(12) Patent Application Publication
(10) Pub. No.: US 2007/0072907 A1
(43) Pub. Date: Mar. 29, 2007

(54) Use of a pyrazole derivative for preparing medicinal products that are useful in the prevention and treatment of dyslipidaemias and of diseases related to dyslipidaemias and/or to obesity

(75) Inventors: Michele Arnone, Ramonville Saint Agne (FR); Jean Marc Herbert, Tournefeuille (FR); Hassan Massoud Heshmati, Philadelphia, PA (US); Philip Janiak, Gif Sur Yvette (FR); Mohammed Bensaid, Juvignac (FR)

Correspondence Address: ROSS J. OEHLER;SANOFI-AVENTIS U.S. LLC, 1041 ROUTE 202-206, MAIL CODE: D303A, BRIDGEWATER, NJ, 08807, US

(73) Assignee: sanofi-aventis, PARIS (FR)

(21) Appl. No.: 11/410,226

(22) Filed: Apr. 24, 2006

Related U.S. Application Data
(63) Continuation of application No. PCT/FR04/02715, filed on Oct. 22, 2004.

(30) Foreign Application Priority Data
Oct. 24, 2003 (FR) 0312553
Dec. 15, 2003 (FR) 0314763
Feb. 5, 2004 (FR) 0401193
Mar. 26, 2004 (FR) 0403252

(51) Int. Cl. A61K 31/454

(52) U.S. Cl. 514/326

(57) ABSTRACT

Use of a pyrazole-derived compound that is an antagonist for cannabinoid CB₁ receptors, alone or combined with another active principal, for preparing medicinal products that are useful in the prevention and treatment of dyslipidaemias and of diseases related to dyslipidaemias and/or to obesity, such as metabolic syndrome, cardiovascular risks and hepatic diseases.

The present invention relates to the use of a pyrazole-derived compound that is an antagonist for cannabinoid CB₁ receptors, for preparing medicinal products that are useful in the prevention and treatment of dyslipidaemias and of diseases related to dyslipidaemias and/or to obesity, such as in particular metabolic syndrome, and also cardiovascular risks and hepatic risks.

Dyslipidaemia is defined by raised triglyceride and LDL-c (Low Density Lipoprotein Cholesterol) levels, by low concentration of HDL-c (High Density Lipoprotein Cholesterol), by an increase in the total cholesterol/HDL-c ratio, and by the presence of small particles of LDL. This dyslipidaemia, often present in obese individuals, is also acknowledged to have an atherogenic profile, i.e. a profile which increases the risk of atheromatous disease.

Obesity is today recognized to be one of the major public health problems. It correlates with a considerable number of cardiovascular diseases, in particular arteriosclerosis, diabetes, of hepatic diseases, in particular non-alcoholic steatohepatitis, of cancers and of respiratory disorders, and it is associated with an increase in mortality rate. The annual costs engendered by the somatic complications of obesity are estimated by the world health organization (WHO) to be a third of the world health budget.

Metabolic syndrome refers to a set of risk factors including dyslipidaemias (low HDL-c level, high triglyceride level), an increase in abdominal circumference/obesity, but also insulin resistance (fasting hyperglycaemia) and arterial hypertension. This syndrome affects several million individuals throughout the world, exposing them to a greater risk of developing diabetes with its complications of renal insufficiency and retinopathy, or of causing a cardiovascular disease such as coronary artery disease, coronary insufficiency, myocardial infarction, angina, atherosclerosis, arteriosclerosis, cerebral stroke, thrombosis, atherothrombosis or glaucoma, or else a hepatic disease such as steatosis, non-alcoholic steatohepatitis, or non-alcoholic fatty liver disease. By improving each parameter of metabolic syndrome, particularly by preventing and treating the elements constituting dyslipidaemia and obesity, the prevention and treatment of metabolic syndrome in patients at risk may contribute to decreasing the appearance of cardiovascular diseases and of type 2 diabetes or else of hepatic diseases.

There is no unified worldwide definition of metabolic syndrome, that given by the National Cholesterol Education Program (NCEP, USA), in the context of an ATP III (Adult Treatment Panel III) group of experts selects the criteria listed in the table below. Patients have a metabolic syndrome when they satisfy at least 3 of the 5 criteria indicated: increase in abdominal circumference/obesity, dyslipidaemia, arterial hypertension, hyperglycaemia.

TABLE 1

| | ATP III |
|---|---|
| Abdominal | Waist: |
| circumference | Men >102 cm |
| | Women >88 cm |
| Lipids | Triglycerides (TG): |
| | ≧150 mg/dl |
| | HDL-c (High Density Lipoprotein |
| | cholesterol) |
| | Men <40 mg/dl |
| | Women <50 mg/dl |
| Blood | ≧130/85 mm Hg |
| pressure | |
| Fasting | ≧110 mg/dl |
| blood sugar | |

According to the present invention, the expression “pyrazole-derived antagonist for cannabinoid receptors, is intended to mean a compound chosen from N-piperidino-5-(4-chlorophenyl)-1-(2,4-dichlorophenyl)-4-methylpyrazole-3-carboxamide, the international common name of which is rimonabant, described in European patent 656354, and N-piperidino-5-(4-bromophenyl)-1-(2,4-dichlorophenyl)-4-ethylpyrazole-3-carboxamide, described in European patent 1150961.

Clinical studies carried out with rimonabant have shown that it acts on food intake from a quantitative and qualitative point of view and reduces the body weight of obese patients (G. Le Fur, 2003, 35, First European Workshop on Cannabinoid Research, Madrid, Spain, 4-5 Apr. 2003 and Heshmati H. M. et al., Obesity Research, 2001, 9 (suppl. 3), 70.

It has now been found that a pyrazole-derived antagonist for cannabinoid CB₁ receptors, chosen from rimonabant and N-piperidino-5-(4-bromophenyl)-1-(2,4-dichlorophenyl)-4-ethylpyrazole-3-carboxamide, exhibits blood lipid-lowering properties (in dyslipidaemic individuals) which may thus contribute to reducing metabolic syndrome in patients exhibiting this syndrome, and decreases the risks of cardiovascular diseases and of hepatic diseases related to obesity and/or to dyslipidaemias.

Thus, according to the present invention, a pyrazole-derived compound that is an antagonist for cannabinoid CB₁ receptors, chosen from rimonabant and N-piperidino-5-(4-bromophenyl)-1-(2,4-dichlorophenyl)-4-ethylpyrazole-3-carboxamide, can be used for preparing medicinal products that are useful for preventing and treating dyslipidaemias and metabolic syndrome, more particularly such a compound that is an antagonist for cannabinoid CB₁ receptors can be used for treating and preventing the risks of cardiovascular diseases and of hepatic diseases related to obesity and/or to dyslipidaemias.

The expression “cardiovascular risks related to dyslipidaemias and/or to obesity” is intended to mean cardiovascular diseases such as: coronary artery disease, coronary insufficiency, atherosclerosis, arteriosclerosis, cerebral stroke, myocardial infarction, angina, thrombosis, atherothrombosis or glaucoma.

The expression “hepatic diseases related to dyslipidaemias and/or to obesity” is intended to mean: hepatic steatosis, non-alcoholic steatohepatitis, non-alcoholic fatty liver disease.

The pharmaceutical compositions according to the present invention contain an effective dose of a pyrazole-derived compound that is an antagonist for cannabinoid CB₁ receptors, chosen from rimonabant and N-piperidino-5-(4-bromophenyl)-1-(2,4-dichlorophenyl)-4-ethylpyrazole-3-carboxamide, and also at least one pharmaceutically acceptable excipient.

Said excipients are chosen, according to the pharmaceutical form and the desired mode of administration, from the usual excipients which are known to those skilled in the art.

In the pharmaceutical compositions of the present invention for oral, sublingual, subcutaneous, intramuscular, intravenous, topical, local, intratracheal, intranasal, transdermal or rectal administration, the active principal can be administered in unit administration form, as a mixture with conventional pharmaceutical excipients, to animals or to human beings, for preventing or treating the disorders or the diseases above.

Suitable unit administration forms comprise the forms for oral administration, such as tablets, soft or hard gelatin capsules, powders, granules and oral solutions or suspensions, the forms for sublingual, buccal, intratracheal, intraocular or intranasal administration and for administration by inhalation, the forms for topical, transdermal, subcutaneous, intramuscular or intravenous administration, the forms for rectal administration, and implants. For topical application, the compounds according to the invention can be used in creams, gels, ointments or lotions.

The forms for oral administration such as gelatin capsules or tablets are preferred.

More particularly, gelatin capsules or tablets containing rimonabant at a dose of between 5 and 50 mg, more particularly the doses of 5 and 20 mg, are preferred.

For the use according to the present invention, a pyrazole-derived antagonist for cannabinoid receptors, chosen from rimonabant and N-piperidino-5-(4-bromophenyl)-1-(2,4-dichlorophenyl)-4-ethylpyrazole-3-carboxamide, can be combined with another active principal chosen from one of the following therapeutic classes:
an angiotensin II AT₁ receptor antagonist, alone or combined with a diuretic;
a converting-enzyme inhibitor, alone or combined with a diuretic or with a calcium antagonist;
a calcium antagonist;
a beta-blocker, alone or combined with a diuretic or with a calcium antagonist;
a blood lipid-lowering agent or a blood cholesterol-lowering agent;
an anti-diabetic agent;
another anti-obesity agent.

Thus, a subject of the present invention is also pharmaceutical compositions containing, in combination, a pyrazole-derived antagonist for cannabinoid CB₁ receptors, chosen from rimonabant and N-piperidino-5-(4-bromophenyl)-1-(2,4-dichlorophenyl)-4-ethylpyrazole-3-carboxamide, and another active principal chosen from one of the following therapeutic classes:
an angiotensin II AT₁ receptor antagonist, alone or combined with a diuretic or with a calcium antagonist;
a converting-enzyme inhibitor, alone or combined with a diuretic;
a calcium antagonist;
a beta-blocker, alone or combined with a diuretic or with a calcium antagonist;
a blood lipid-lowering agent or a blood cholesterol-lowering agent;
an anti-diabetic agent;
another anti-obesity agent.

The expression “angiotensin II AT₁ receptor antagonist” is intended to mean a compound such as candesartan cilexitil, eprosartan, irbesartan, losartan potassium, olmesartan medoxomil, telmisartan or valsartan, it being possible for each of these compounds to itself be combined with a diuretic such as hydrochlorothiazide.

The expression “converting-enzyme inhibitor” is intended to mean a compound such as alacepril, benazepril, captopril, cilazapril, enalapril, enalaprilat, fosinopril, imidapril, lisinopril, moexipril, perindopril, quinapril, ramipril, spirapril, temocapril, trandolapril or zofenopril, it being possible for each of these compounds to itself be combined with a diuretic such as hydrochlorothiazide or indapamide or with a calcium antagonist such as amlodipine, diltiazem, felodipine or verapamil.

The term “calcium antagonist” is intended to mean a compound such as amlodipine, aranidipine, benidipine, bepridil, cilnidipine, diltiazem, efonidipine hydrochloride ethanol, fasudil, felodipine, isradipine, lacidipine, lercanidipine hydrochloride, manidipine, mibefradil hydrochloride, nicardipine, nifedipine, nilvadipine, nimodipine, nisoldipine, nitrendipine, terodiline or verapamil.

The term “beta-blocker” is intended to mean a compound such as acebutolol, alprenolol, amosulalol, arotinolol, atenolol, befunolol, betaxolol, bevantolol, bisoprolol, bopindolol, bucumolol, bufetolol, bunitrolol, butofilolol, carazolol, carteolol, carvedilol, cloranolol, epanolol, esmolol, indenolol, labetalol, landiolol, levobunolol, levomoprolol, mepindolol, metipranolol, metoprolol, nadolol, nebivolol, nifenalol, nipradilol, oxprenolol, penbutolol, pindolol, propanolol, salmeterol, sotalol, talinolol, tertatolol, tilisolol, timolol, xamoterol or xibenolol.

The expression “blood lipid-lowering agent” or “blood cholesterol-lowering agent” is intended to mean a compound chosen from fibrates such as alufibrate, beclobrate, bezafibrate, ciprofibrate, clinofibrate, clofibrate, etofibrate, fenofibrate; statins (HMG-CoA reductase inhibitors) such as atorvastatin, fluvastatin sodium, lovastatin, pravastatin, rosuvastatin or simvastatin, or a compound such as acipimox, aluminium nicotinate, azacosterol, cholestyramine, dextrothyroxine, meglutol, niceritrol, nicoclonate, nicotinic acid, beta-sitosterine or tiadenol. More particularly, a subject of the present invention is a pharmaceutical composition containing, in combination, rimonabant, and atorvastatin or pravastatin, or preferably rimonabant and simvastatin.

The term “anti-diabetic agent” is intended to mean a compound belonging to one of the following classes: sulphonylureas, biguanidines, alpha-glucosidase inhibitors, thiazolidine diones, metiglinides, such as acarbose, acetohexamide, carbutamide, chlorpropamide, glibenclamide, glibornuride, gliclazide, glimepiride, glipizide, gliquidone, glisoxepide, glybuzole, glymidine, metahexamide, metformin, miglitol, nateglinide, pioglitazone, repaglinide, rosiglitazone, tolazamide, tolbutamide, troglitazone or voglibose.

The term “another anti-obesity agent” is intended to mean a compound such as amfepramone, benfluorex, benzphetamine, indanorex, mazindole, mefenorex, methamphetamine, D-norpseudoephedrine or another antagonist for cannabinoid CB₁ receptors.

Most particularly, a subject of the present invention is a pharmaceutical composition containing, in combination, rimonabant and an angiotensin II AT₁ receptor antagonist, in particular irbesartan, losartan or valsartan. More particularly, a subject of the present invention is a pharmaceutical composition containing, in combination, rimonabant and irbesartan, or N-piperidino-5-(4-bromophenyl)-1-(2,4-dichlorophenyl)-4-ethylpyrazole-3-carboxamide and irbesartan, and also a pharmaceutical composition containing, in combination, rimonabant, irbesartan and hydrochlorothiazide, or N-piperidino-5-(4-bromophenyl)-1-(2,4-dichlorophenyl)-4-ethylpyrazole-3-carboxamide, irbesartan and hydrochlorothiazide.

According to another particular embodiment, a subject of the present invention is a pharmaceutical composition containing, in combination, rimonabant and simvastatin.

According to another aspect of the invention, the pyrazole-derived antagonist for cannabinoid receptors, chosen from rimonabant and N-piperidino-5-(4-bromophenyl)-1-(2,4-dichlorophenyl)-4-ethylpyrazole-3-carboxamide, and the other combined active principal can be administered simultaneously, separately or in a manner spread out over time.

The term “separate use” is intended to mean the administration, at the same time, of the two compounds of the composition according to the invention, each included in a distinct pharmaceutical form.

The expression “use spread out over time” is intended to mean the successive administration of the first compound of the composition according to the invention, included in a pharmaceutical form, and then of the second compound of the composition according to the invention, included in a distinct pharmaceutical form.

In the case of this “use spread out over time”, the period of time that elapses between the administration of the first compound of the composition according to the invention and the administration of the second compound of the same composition according to the invention does not generally exceed 24 hours; it may be longer if one or other of the compounds is provided in a pharmaceutical form allowing, for example, weekly administration.

The pharmaceutical forms, comprising either just one of the compounds constituting the composition according to the invention or the combination of the two compounds or, where appropriate, of three compounds, which can be used in the various types of uses described above, may, for example, be suitable for oral, nasal, parenteral or transdermal administration.

Thus, in the case of a “separate use” and of a “use spread out over time”, two distinct pharmaceutical forms may be intended for the same route of administration or for a different route of administration (oral and transdermal or oral and nasal or parenteral and transdermal, etc.).

The invention therefore also relates to a kit containing a pyrazole-derived antagonist for cannabinoid CB₁ receptors, chosen from rimonabant and N-piperidino-5-(4-bromophenyl)-1-(2,4-dichlorophenyl)-4-ethylpyrazole-3-carboxamide, and another active principal or, where appropriate, two combined active principals, in which kit said pyrazole-derived antagonist for cannabinoid CB₁ receptors and said active principal or, where appropriate, two combined active principals are in distinct compartments and in similar or different packagings, and are intended to be administered simultaneously, separately or in a manner spread out over time.

### EXAMPLE 1

### Action of Rimonabant on Blood Lipid Levels in Obese Mice

The effect of a long-term (2 months) treatment with rimonabant was studied in mice having an established obesity.

The study was carried out in mice receiving either a normal diet or a fatty diet. Obesity developed in the mice receiving a fatty diet and it became stabilized after 5 months. The mice were then divided up into 3 groups:

group 1: maintenance of the fatty diet and oral treatment for 2 months with rimonabant at 10 mg/kg/day, in water with 0.1% of Tween 80 (vehicle);

group 2: maintenance of the fatty diet and administration of the vehicle (water+0.1% of Tween 80);

group 3: return to a normal diet and administration of the vehicle (water+0.1% of Tween 80);

group 4 consists of mice receiving a normal diet and the vehicle from the start.

After 5 months of fatty diet, the mice showed a 46% weight gain and a marked increase in blood leptin, insulin, glucose and total cholesterol levels.

For these obese mice, the HDLc (high density lipoprotein cholesterol) and LDLc (low density lipid cholesterol) levels were measured and an increase in these levels, accompanied by a decrease in the HDLc/LDLc ratio, was observed.

After 2 months of treatment with rimonabant, the weight of the mice in group 1 decreased by 34.5±0.8 g, i.e. in the same proportions as that of the mice in group 3 that were returned to a normal diet (33.7±0.6 g).

Likewise, after 2 months of treatment with rimonabant, the mice in group 1 showed a decrease in leptin, insulin and glucose blood levels; similarly, these levels were reduced for the mice in group 3.

The data measured in the assays for triglycerides and for cholesterol lipoproteins are given in the tables below:

TABLE 2
Distribution of triglycerides and of cholesterol in the obese
mice after 2 months of treatment or a return to a normal diet

| | | | Total cholesterol |
|---|---|---|---|
| Group | n | Triglycerides mmol/L | g/L |
| 1 | 28 | 0.74 ± 0.04 | 1.73 ± 0.07# |
| 2 | 74 | 0.93 ± 0.03* | 2.04 ± 0.04* |
| 3 | 9 | 0.72 ± 0.03 | 1.21 ± 0.05 |
| 4 | 31 | 0.78 ± 0.03 | 1.15 ± 0.02 |

*p < 0.001 with respect to other groups
#p < 0.01 with respect to groups 3 and 4

TABLE 3
Distribution of the cholesterol fractions in the obese
mice after 2 months of treatment or return to a normal
diet

| | | | | HDLc/total | LDLc/total | |
|---|---|---|---|---|---|---|
| Group | n | HDLc mg/dL | LDLc mg/dL | cholesterol % | cholesterol % | HDLc/LDLc % |
| 1 | 28 | 107.8 ± 4.5* | 9.3 ± 0.5 | 62.2 ± 0.5# | 5.5 ± 0.3* | 12.4 ± 0.8# |
| 2 | 74 | 115.7 ± 2.0* | 16.2 ± 0.8* | 57.1 ± 0.4* | 7.7 ± 0.2 | 7.9 ± 0.2* |
| 3 | 9 | 78.2 ± 4.5 | 7.3 ± 0.6 | 64.3 ± 2.0# | 6.1 ± 0.6* | 11.5 ± 1.3 |
| 4 | 31 | 77.7 ± 1.8 | 8.1 ± 0.4 | 67.5 ± 0.8 | 7.1 ± 0.3 | 10.3 ± 0.6 |
| | | *p < 0.001 | *p < 0.001 | *p < 0.001 | *p < 0.05 | *p < 0.001 |
| | | with respect | with respect | with | with | with |
| | | to groups 3 | to other | respect to | respect to | respect to |
| | | and 4 | groups | other | group 2 | other |
| | | | | groups | | groups |
| | | | | #p < 0.05 | | #p < 0.05 |
| | | | | with | | with |
| | | | | respect to | | respect to |
| | | | | group 4 | | group 4 |

n: number of animals.

According to Tables 2 and 3, it is seen that the treatment with rimonabant, as administered to the animals in group 1, corrects the hypertriglyceridaemia observed in the animals subjected to a fatty diet (group 2).

According to Tables 2 and 3, it is seen that the treatment with rimonabant makes it possible to lower the total cholesterol level, but not to normalize it; this same treatment makes it possible to normalize the LDLc level, the consequence of which is to increase the HDLc/LDLc ratio.

It may be concluded that the treatment with rimonabant produces favourable modifications in the plasma lipid profile, although the mice are kept on a fatty diet: specifically, although the decrease in the total cholesterol level is modest, the treatment normalizes the triglyceride and the LDLc level while at the same time keeping the “protective” level of HDLc high, and thus the HDLc/LDLc ratio is greater in the obese animals which followed the treatment with rimonabant than in the obese animals treated with the vehicle alone, whether they were subjected to a fatty or normal diet.

### EXAMPLE 2

### Clinical Action of Rimonabant on the Lipid Parameters, after 4 Weeks of Treatment

A clinical trial was carried out for 4 weeks on 287 obese patients whose body mass index (BMI) was between 30 and 40. After having received the placebo for 2 weeks, the patients were randomized so as to receive doses of 5, 10 or 20 mg/day of rimonabant or the placebo.

There was a check-up visit 4 weeks after the end of the treatment.

For the duration of the study, the patients were asked to follow a low-calorie diet (deficit of 500 kcal/day).

The results observed at the end of the treatment are given in the table below:

TABLE 4

| | | Decrease | | Blood | |
|---|---|---|---|---|---|
| | Weight | in | Increase | glucose | Improvement |
| Treatment | loss | triglycerides | in HDLc | level | in metabolic |
| group | (kg) | ≧10% | ≧10% | mmol/l | syndrome |
| Placebo | 1.1 | 34% | 34% | +0.26 | 19% |
| 5 mg/d | 3.4* | 36% | 43% | −0.09** | 42% |
| 10 mg/d | 3.7* | 34% | 45% | −0.02*** | 39% |
| 20 mg/d | 4.5* | 51% | 44% | +0.03 | 21% |

*p < 0.005 with respect to the placebo group.
**p < 0.01 with respect to the placebo group.
***p < 0.05 with respect to the placebo group.

It is noted that the decrease in weight in the individuals treated with rimonabant is accompanied by a tendency for the triglycerides to decrease and for the HDLcs to increase. In parallel, the blood glucose level remains stable or decreases discretely for the patients in the groups treated with rimonabant, whereas the blood glucose level of the patients in the placebo group increases.

The influence, in the obese patients, of the treatment with rimonabant on various biological parameters, taken into account in evaluating the metabolic syndrome, as defined by ATP III was thus observed. It results in a tendency for the metabolic syndrome to improve in the patients treated with rimonabant.

### EXAMPLE 3

### Clinical Action of Rimonabant on the Lipid Parameters and Prevalence of Metabolic Syndrome, after 12 Months of Treatment

The Rio Lipids clinical study, carried out for 12 months in 1036 obese individuals with dyslipidaemias, compares the effect of rimonabant at a dose of 20 mg, versus a placebo product, in weight reduction, improvement in the lipid parameters and prevalence of metabolic syndrome. The treated group and the placebo group are subjected to a low-calorie diet.

The individuals treated with rimonabant at a dose of 20 mg for 12 months show a weight loss that is 6.3±0.5 kg greater than that observed in the placebo group (p≦0.001).

In this same population, the increase in the HDLc level exceeds that observed in the placebo group by 11.3±1.7%.

The decrease in the triglyceride level in the treated group exceeds that of the placebo group by 12.2±3.7 (p≦0.001).

An increase in adiponectin from 5.8±2.7 μg/ml to 8.2±4.2 μg/ml at the end of the year of treatment with rimonabant at a dose of 20 mg was also noted.

The adiponectin reflects the insulin-resistant state: the variation in its level is inversely proportional to that of the insulin resistance. Thus, in the present case, the increase in the adiponectin level indicates a decrease in the level of insulin resistance.

Finally, in the group treated with rimonabant, 60.2% of the patients stopped showing the metabolic syndrome characteristics, whereas the proportion was 40.4% in the placebo group (p<0.001).

Thus, since the placebo group was subjected to the same low-calorie diet as the treated group, it appears that rimonabant has a specific effect on the decrease in metabolic syndrome.

Thus, in a clinical trial lasting one year, the action of rimonabant on the parameters of dyslipidaemia, on several elements constituting metabolic syndrome and on the metabolic syndrome itself is observed.

### EXAMPLE 4

### Effect of Rimonabant on Steatosis and Steatohepatitis in Obese Rats

The effect of rimonabant on steatosis and steatohepatitis was studied in obese fa/fa Zucker rats.

Obese fa/fa Zucker rats, in which the leptin receptors are functionally defective, show obesity related to hyperleptinaemia, hyperinsulinaemia and dyslipidaemia and exhibit steatohepatitis.

For this study, 3 groups are formed:
thin rats/vehicle group: thin Zucker rats treated with the vehicle (water and 0.1% Tween 80);
obese rats/vehicle group: obese fa/fa Zucker rats treated with the vehicle (water and 0.1% Tween 80);
obese rats/rimonabant group: obese fa/fa Zucker rats treated orally for 2 months with rimonabant at 30 mg/kg/day, in the vehicle (water and 0.1% Tween 80).

After 2 months of treatment, the body weight and the weight of the liver are measured for each rat, and a histopathological analysis of the fat load in the livers is carried out.

The results show that the liver weight/body weight ratio is 41% higher in the obese rats/vehicle group compared with the thin rats/vehicle group.

The treatment of the obese fa/fa rats with rimonabant decreases by 80% the increase in the liver weight/body weight ratio, observed in the rats of the obese/vehicle group, to reach a ratio having a value comparable to that observed in the thin rats/vehicle group (Table 5).

TABLE 5
Decrease in the liver weight/body weight ratio in the obese
fa/fa Zucker rats after treatment for 2 months with rimonabant

| | Number of | Liver weight/body |
|---|---|---|
| Animals/treatment | animals | weight ratio (%) |
| Thin rats/vehicle | 12 | 3.48 ± 0.13 |
| Obese rats/vehicle | 12 | 4.92 ± 0.14** |
| Obese rats/rimonabant | 10 | 3.86 ± 0.07^{##} |

**p < 0.01 compared to the thin rats/vehicle group
^{##}p < 0.01 compared to the obese rats/vehicle group.

The histopathological analyses of the fat overload in the livers show that the livers of the obese rats/vehicle group exhibit a substantial fat overload. The treatment of these rats with rimonabant induces a disappearance of this fat overload. The sections of livers from the obese rats/rimonabant group show a histological profile comparable to that of the sections of livers from the thin rats/vehicle group. These data show that the treatment with rimonabant greatly decreases the fat overload in the liver of obese fa/fa rats, i.e. the hepatic steatosis.

### EXAMPLE 5

### Action of Rimonabant and of Irbesartan on the Plasma Lipid Levels in Obese Rats

The effect of rimonabant alone or in combination with irbesartan was studied in obese fa/fa Zucker rats. For this study, 7 groups were formed:

group 1: thin Zucker rats treated with the vehicle,

group 2: obese fa/fa Zucker rats treated with the vehicle,

group 3: obese fa/fa Zucker rats treated with rimonabant at 1 mg/kg/day,

group 4: obese fa/fa Zucker rats treated with rimonabant at 3 mg/kg/day per os,

group 5: obese fa/fa Zucker rats treated with irbesartan at 3 mg/kg/day per os,

group 6: obese fa/fa Zucker rats treated with rimonabant at 1 mg/kg/day per os and irbesartan at 3 mg/kg/day per os,

group 7: obese fa/fa Zucker rats treated with rimonabant at 3 mg/kg/day per os and irbesartan at 3 mg/kg/day per os.

After 3 months of treatment, the rimonabant+irbesartan combination significantly reduces the plasma cholesterol and triglyceride levels in the obese fa/fa Zucker rats.

A synergistic effect between the rimonabant and the irbesartan is noted. Administration of the 2 compounds combined improves the HDLc/LDLc ratio for the treated animals.

### EXAMPLE 6

### Pharmaceutical Composition

For administration to the patients, the rimonabant is formulated in pharmaceutical compositions which are prepared by wet granulation.

| CONSTITUENTS | | | | |
|---|---|---|---|---|
| Micronized rimonabant | 20.0 | mg | 5.0 | mg |
| Corn starch | 67.50 | mg | 45.0 | mg |
| Lactose monohydrate | 111.66 | mg | 82.8 | mg |
| Povidone* | 5.25 | mg | 3.5 | mg |
| Sodium croscarmellose | 18.75 | mg | 12.5 | mg |
| Sodium lauryl sulphate | 0.34 | mg | 0.2 | mg |
| Microcrystalline cellulose | 75.0 | mg | 50.0 | mg |
| Magnesium stearate | 1.50 | mg | 1 | mg |
| Tablet with a final weight of | 300 | mg | 200 | mg |

*Povidone is defined in the European Pharmacopoeia as follows: poly(1-(2-oxo-1-pyrrolidinyl)ethylene), and consists of linear polymers of 1-vinylpyrrolidin-2-one.

The tablets are preferably coated using a suitable excipient.

1. Use of a compound that is an antagonist for cannabinoid CB₁ receptors, chosen from rimonabant and N-piperidino-5-(4-bromophenyl)-1-(2,4-dichlorophenyl)-4-ethylpyrazole-3-carboxamide, for preparing medicinal products that are useful in the treatment and prevention of diseases related to obesity and/or to dyslipidaemias, chosen from metabolic syndrome, cardiovascular risks and hepatic diseases.

2. Use according to claim 1, for preventing and treating dyslipidaemias.

3. Use according to claim 1, for preventing and treating metabolic syndrome.

4. Use of a compound according to claim 1, for preventing and treating cardiovascular risks related to obesity and/or to dyslipidaemias.

5. Use of a compound according to claim 1, for preventing and treating hepatic diseases related to obesity and/or to dyslipidaemias.

6. Use according to claim 5, for preventing and treating steatosis and/or non-alcoholic steatohepatitis.

7. Use according to claim 1, in which the antagonist for cannabinoid CB₁ receptors is combined with another active principal chosen from one of the following therapeutic classes:
an angiotensin II AT₁ receptor antagonist, alone or combined with a diuretic or with a calcium antagonist;
a converting-enzyme inhibitor, alone or combined with a diuretic;
a calcium antagonist;
a beta-blocker, alone or combined with a diuretic or with a calcium antagonist;
a blood lipid-lowering agent or a blood cholesterol-lowering agent;
an anti-diabetic agent;
another anti-obesity agent.

8. Use according to claim 1, in which the compound that is an antagonist for cannabinoid CB₁ receptors is rimonabant.

9. Use according to claim 8, in which the rimonabant is used at a dose of between 5 mg and 50 mg.

10. Pharmaceutical composition containing, in combination, a compound that is an antagonist for cannabinoid CB₁ receptors, chosen from rimonabant and N-piperidino-5-(4-bromophenyl)-1-(2,4-dichlorophenyl)-4-ethylpyrazole-3-carboxamide, and another active principal chosen from one of the following therapeutic classes:
an angiotensin II AT₁ receptor antagonist, alone or combined with a diuretic or with a calcium antagonist;
a converting-enzyme inhibitor, alone or combined with a diuretic;
a calcium antagonist;
a beta-blocker, alone or combined with a diuretic or with a calcium antagonist;
a blood lipid-lowering agent or a blood cholesterol-lowering agent;
an anti-diabetic agent;
another anti-obesity agent.

11. Pharmaceutical composition according to claim 10, containing, in combination, a compound that is an antagonist for cannabinoid CB₁ receptors, chosen from rimonabant and N-piperidino-5-(4-bromophenyl)-1-(2,4-dichlorophenyl)-4-ethylpyrazole-3-carboxamide, and an angiotensin II AT₁ receptor antagonist, alone or combined with a diuretic.

12. Pharmaceutical composition according to claim 11, containing, in combination, rimonabant and irbesartan.

13. Pharmaceutical composition according to claim 11, containing, in combination, rimonabant, irbesartan and hydrochlorothiazide.

14. Pharmaceutical composition according to claim 10, containing, in combination, an antagonist for cannabinoid CB₁ receptors, chosen from rimonabant and N-piperidino-5-(4-bromophenyl)-1-(2,4-dichlorophenyl)-4-ethylpyrazole-3-carboxamide, and a converting-enzyme inhibitor, alone or combined with a diuretic.

15. Pharmaceutical composition according to claim 10, containing, in combination, an antagonist for cannabinoid CB₁ receptors, chosen from rimonabant and N-piperidino-5-(4-bromophenyl)-1-(2,4-dichlorophenyl)-4-ethylpyrazole-3-carboxamide, and a calcium antagonist.

16. Pharmaceutical composition according to claim 10, containing, in combination, an antagonist for cannabinoid CB₁ receptors, chosen from rimonabant and N-piperidino-5-(4-bromophenyl)-1-(2,4-dichlorophenyl)-4-ethylpyrazole-3-carboxamide, and a beta-blocker, alone or combined with a diuretic or with a calcium antagonist.

17. Pharmaceutical composition according to claim 10, containing, in combination, an antagonist for cannabinoid CB₁ receptors, chosen from rimonabant and N-piperidino-5-(4-bromophenyl)-1-(2,4-dichlorophenyl)-4-ethylpyrazole-3-carboxamide, and a blood lipid-lowering agent or a blood cholesterol-lowering agent.

18. Pharmaceutical composition according to claim 10, containing, in combination, an-antagonist for cannabinoid CB₁ receptors, chosen from rimonabant and N-piperidino-5-(4-bromophenyl)-1-(2,4-dichlorophenyl)-4-ethylpyrazole-3-carboxamide, and an anti-diabetic agent.

19. Pharmaceutical composition according to claim 18, containing, in combination, rimonabant and simvastatin.

20. Pharmaceutical composition according to claim 10, containing, in combination, an antagonist for cannabinoid CB₁ receptors, chosen from rimonabant and N-piperidino-5-(4-bromophenyl)-1-(2,4-dichlorophenyl)-4-ethylpyrazole-3-carboxamide, and another anti-obesity agent.

21. Kit containing a pyrazole-derived antagonist for cannabinoid CB₁ receptors, chosen from rimonabant and N-piperidino-5-(4-bromophenyl)-1-(2,4-dichlorophenyl)-4-ethylpyrazole-3-carboxamide, and another active principal chosen from the active principals of one of the following therapeutic classes:
an angiotensin II AT₁ receptor antagonist, alone or combined with a diuretic or with a calcium antagonist;
a converting-enzyme inhibitor, alone or combined with a diuretic;
a calcium antagonist;
a beta-blocker, alone or combined with a diuretic or with a calcium antagonist;
a blood lipid-lowering agent or a blood cholesterol-lowering agent;
an anti-diabetic agent;
another anti-obesity agent.
